(19) United States
(12) Patent Application Publication
(10) Pub. No.: US 2011/0136758 A1
(43) Pub. Date: Jun. 9, 2011

(54) CARBOHYDRATE COMPOSITIONS FROM BASIDIOMYCETE FUNGI AS BIOCIDAL AGENTS ACTIVE AGAINST PATHOGENS

(76) Inventor: Bryan Hiromoto, Puunene, HI (US)

(21) Appl. No.: 11/997,727

(22) PCT Filed: Aug. 1, 2006
(86) PCT No.: PCT/US06/30009

§ 371 Date: Feb. 28, 2011

Related U.S. Application Data
(60) Provisional application No. 60/704,824, filed on Aug. 1, 2005.

(51) Int. Cl. A61K 31/715; C12P 1/02; A01N 43/04; C07H 1/00; A01P 3/00; A61P 31/04; A61P 31/10; A01P 15/00; A01P 5/00; A61P 33/00; C12N 1/00

(52) U.S. Cl. 514/54; 435/171; 536/123.1; 435/254.1

(57) ABSTRACT

The invention provides biologically active compositions comprising oligosaccharides, and which are produced by growing a fungal culture, and are at least partially purified for use. The compositions of the invention have antibacterial, antifungal and nematicidal activity, and are thus useful to reduce the impact of such pathogens on growing plants, to reduce the occurrence of such pathogens on surfaces and in substances, and to treat infections caused by such pathogens in animals and humans. The invention also provides methods for producing such compositions from certain fungal cultures.

### RELATED APPLICATIONS

This application is related to U.S. Provisional Application No. 60/704,824, filed Aug. 1, 2005, the contents of which are incorporated herein by reference.

### TECHNICAL FIELD

The invention relates to novel compositions that have activity against microbes and other pathogens; the compositions are produced by certain species of Basidiomycete fungi. It provides carbohydrate-containing compositions that have biocidal activity against a variety of pathogenic organisms including nematodes, fungi and bacteria. It also provides conditions for growing Basidiomycete fungi to enhance production of the bioactive substances, as well as methods of using these compositions to protect plants from damage caused by plant pathogens and in other applications.

### BACKGROUND ART

Some species of mushrooms are well known to be toxic or hallucinogenic. Several species are reported to produce metabolites having biocidal activities against nematodes, or against other fungal species, as well as antibacterial activity against some human pathogenic bacteria. For example, Coletto, et al., report activity against both Gram positive and Gram negative human pathogenic bacteria with filtrates from cultures of certain Basidiomycetes, including Laetiporus sulphureus. M. A. B. Coletto, et al., “Basidiomiceti in relazione all'antibiosi. Nota XI. Attivita antibatterica e antifungina di 25 nuova ceppi”, Allionia, vol. 35, 95-101 (1997) (Abstract in English). Coletto also tested some fungal strains for activity against phytopathogenic fungi. (Id.) A summary of some of the bioactive compounds produced by various fungi is provided in the Doctoral Thesis of Loreto Robles Hernández, University of Idaho, March 2005. Most of these compounds that have been characterized are either peptides or terpenoid compounds: few bioactive carbohydrates from fungi have been reported. Relatively little is known about fungal metabolites that have activity against plant pathogens, too, although U.S. Pat. No. 6,517,851 and U.S. Pat. No. 6,048,714 report the nematicidal activity of certain compositions produced by culturing fungi on an oil-containing medium, and at least one report indicates that Laetiporus sulphureus can produce cinnamaldehyde, which is known to have pesticidal activity against certain insects and diseases that injure plants. S. Rapior, et al., “Volatile composition of Laetiporus sulphureus”, Cryptogamie Mycologie, vol. 21, 67-72 (2000): Abstract in English; and CINNAMITE® miticide specimen label, disclosing activity against mites, aphids, and powdery mildew. In addition to these reports, various studies have disclosed the structures of volatiles that provide flavor and odor of certain Basidiomycetes, and have characterized some polysaccharides produced by these fungi. See e.g. S. Wu, et al., “Characteristic Volatiles from Young and Aged Fruiting Bodies of Wild Polyporus sulfurous”, J. Agricultural and Food Chem. 2005, 53, 4524-28; and G. Alquini, et al., “Polysaccharides from the fruit bodies of basidiomycete Laetiporus sulphureus (Bull.: Fr.) Murr”, FEMS Microbiology Lett. 230, 47-52 (2004). Because of the wide variety of bioactive compounds produced by various fungi, they should be a rich source of natural products having activity against plant pathogens.

The Basidiomycetes are a large family of very diverse fungi, including edible mushrooms, toxic mushrooms, puffballs, and even microscopic species; many have shown at least some biological activity. Ganoderma lucidum is a Basidiomycete mushroom known in oriental medicine for its production of substances with potent immuno-modulating action. Bao, Wang, Dong, Fang, & Li, “Structural features of immunologically active polysaccharides from Ganoderma lucidum”, Phytochemistry, 59, 175-81 (2002); Su et al., “Fungal mycelia as the source of chitin and polysaccharides and their applications as skin substitutes,” Biomaterials, 18, 1169-74 (1997). In addition to the immuno-modulating compounds, some Ganoderma species have demonstrated bactericidal activity. Crude extracts of Ganoderma species G. lucidum, G. pfeifferi, and G. resinaceum) inhibited growth of Bacillus subtilis. Suay et al., “Screening of basidiomycetes for antimicrobial activities”, Antonie van Leeuwenhoek, 78, 129-39 (2000). No oligosaccharides derived from these fungi having biocidal activity have been reported, however.

Limited antifungal activity has been reported in metabolites from Basidiomycetes. The oyster mushroom, Pleurotus ostreatus inhibited growth of Aspergillus niger. Gerasimenya et al., “Antimicrobial and antitoxical action of edible and medicinal mushroom Pleurotus ostreatus (Jacq.:Fr.) Kumm. extracts”, Int. J. Med. Mushrooms, 4, 106 (2002). A member of the Poriales, Gloeophyllum sepiarium had antagonistic activity against S. cerevisiae and Aspergillus fumigatus (Suay et al., 2000). Ganoderma applanatum was reported to prevent growth of rhizomorphs of Armillaria luteobubalina. Perch, M., “In vitro interactions between Armillaria luteobubalina and other wood decay fungi, Mycol. Res. 94, 753-61 (1990). Thus, there are a number of reports of Basidiomycete metabolites causing suppression of growth of heterologous fungi, but few reports of actual fungicidal activity, or of utility to protect cultivated plants or fungi from fungal damage.

The nematicidal activity of mushroom metabolites has been reported in very rare cases and is usually attributed to parasitism. However, nematicidal activity produced by certain fungal cultures has recently been reported in U.S. Pat. No. 6,517,851 and U.S. Pat. No. 6,048,714. In addition, a commercial fertilizer product known as “Maui LCF” (LCF stands for ‘liquid compost factor’) has been said to induce tolerance to nematodes in some plants. Maui LCF is a fertilizer that is produced by growing L. sulphureus on a medium consisting of plant-derived materials including pineapple, papaya and sugar cane materials. It enhances plant health and provides a means to reduce dependence on pesticides. Economic Development of Hawaii, ‘Activities Underway in 2003”, available online at http://www.epa.gov/oppbppd1/PESP/publications/vol6se/IIIF-edah.htm. This fluid from composting of plant materials in an aqueous milieu is heated to denature proteins, then filtered to remove solids, and is sold as a brown solution without further purification. Maui LCF is applied to the soil where plants or seeds have been, or will be, introduced, as a fertilizer or fertilizer additive; it provides nutrients and plant growth regulatory compounds, but is not classified as a pesticide. There have been some reports of apparent fungicidal activity with Maui LCF: in light of laboratory evidence that heating destroys the fungicidal activity of Maui LCF, though, the observations of fungicidal activity are attributed to ‘undercooking’ of batches of Maui LCF.

Thus, Basidiomycete fungi produce a variety of molecules with useful biological activity, but have not been extensively exploited as a source of plant-protective agents. Furthermore, few known bioactives from fungi have activity against plant pathogens, especially plant bacteria. While the destructive impact of insects and fungal diseases on agronomic and ornamental plants is much larger, bacterial diseases of plants cause billions of dollars worth of damage every year, and there are few good treatments for them. Antibacterials active against plant bacteria, and especially natural product-derived antibacterials, would be of great value.

The present invention provides novel antibacterial carbohydrate or oligosaccharide compounds and compositions derived from Basidiomycete fungi having utility for protection of plants. It also provides a compost tea made from the culture fluid of a Basidiomycete fungus: the composition of this tea is not fully characterized, but it protects plants and their fruit or vegetable products from injury caused by plant pathogens, and accelerates recovery from injury caused by pathogens. These compounds and compositions provide biocidal activity against plant pathogens, including bacteria, fungi and nematodes, and are especially valuable because of their activity as antibacterial agents that suppress or kill phytopathogenic bacteria. The antibacterial compositions also have activity as a wood preservative that deters damage to wood caused by termites and/or fungi.

### DISCLOSURE OF THE INVENTION

The invention provides novel biocidal oligosaccharide- or carbohydrate-containing antibacterial compositions produced by certain species of Basidiomycete fungi. In particular, it provides novel compositions containing carbohydrate substances that are produced by some Basidiomycete species including Ganoderma lucidum and Laetiporus sulphureus and methods for using them to protect cultivated plants and plant products. It also provides methods for producing such compositions by growing a Basidiomycete fungal culture, including methods for improving the yield of the bioactive species during large-scale production.

The invention also provides a compost tea produced by Basidiomycete cultures. The tea may comprise multiple active factors having plant protectant activities and other useful biocidal activities against pathogens, and may be partially purified by methods such as sterilization or filtration to remove cellular materials, or by other concentration methods such as reverse osmosis. The invention also provides methods for producing the compositions described herein that increase the activity of the product, including preferred culture media and growing conditions.

While a Laetiporus-derived material known as Maui LCF (LCF stands for liquid compost factor and is sold as a crude brown solution collected from the culture medium of L. sulphureus), has been produced and used for its plant growth regulatory and plant nutrient (fertilizer) effects, it has now been found that a modified process for growing and processing the culture fluids from the same and other Basidiomycete fungi produces a product that unexpectedly provides control of phytopathogens and other pathogens while it avoids overstimulation of plant growth, which may be undesirable. The present compositions also retain these activities without causing discoloration of plants and/or plant products that can occur with the crude compositions of the prior art. The new compositions have reduced side effects, and they reduce injury to plants or plant products when used against phytopathogens, improve yields and crop quality, and accelerate recovery of an injured plant or fruit after injury caused by pathogens including fungi, bacteria and nematodes. The compositions may be administered directly to the foliage and/or plant product and/or fruit or vegetable to be treated, or to the soil adjacent such plants, or to the roots of the plant.

Certain compositions of the invention provide antibacterial activity against microorganisms that can cause diseases in higher animals, thus the compositions can be used to kill or prevent growth of such non-plant pathogens. For use against non-plant pathogens, the compositions may be administered to a mammal to be treated, or they may be applied to a surface to be treated using means known in the art. They can, for example, be used in vivo, by administering an effective amount of a composition of the invention to an animal to be treated for or protected from a pathogen. Alternatively, the compositions can be applied to a surface suspected of being contaminated with such non-plant pathogen, or applied to or admixed with a liquid such as standing water that can serve as a growing medium for a pathogen. Also, they can be applied to an insect or other carrier that can act as a vector for the pathogen, or to the locus of such insect or other carrier. The latter approach reduces the likelihood that an animal will become infected, by reducing the number and/or viability of the pathogen to which the animal is exposed, whether the exposure involves direct contact with the pathogen or delivery of the pathogen by a vector.

The compositions of the invention may include multiple active species, and thus may act by multiple mechanisms. The invention thus provides novel antibacterial compositions derived from Basidiomycete culture fluid that are active to stop the growth of a variety of different plant-dwelling bacteria and other pathogens, or to induce systemic resistance of a plant toward one or more pathogens, or to induce plant tolerance to injury caused by a pathogen. The culture fluid can be used in crude form, or an active composition can be obtained by applying known purification methods to the culture fluid. The activity of the compositions against plant-dwelling bacteria is believed to be novel, and the partially purified compositions comprising antibacterial compounds are also novel.

Culture fluids from G. lucidum and L. sulphureus were also observed to have fungicidal activity against most fungi tested. This activity was bench stable (stable at ambient temperatures when exposed to air), but heat labile, suggesting that this activity is of a different nature than the bactericidal activity. A few reports have shown that some species of mushrooms have fungicidal activity on several plant pathogenic fungi. However, the fungicidal activity of G. lucidum or L. sulphureus has not been reported previously against the pathogens used in this study.

In addition, the bactericidal effects of the compositions of the invention can be used to preserve wood. When applied to wood that is subject to termite damage, the compositions may deter consumption of the wood by the termite (antifeedant activity) or cause injury or death to termites that consume the wood (termiticidal activity). Without being bound by theory of operation, this effect may result from toxicity of the composition to symbiotic bacteria required by the termite for digesting cellulose. Thus the instant compositions provide a natural protectant for wood that deters damage by termites and may also deter damage caused by certain wood-rotting fungi. The compositions are biodegradable enough to provide a safer alternative to potentially hazardous arsenic, copper and chromium compounds typically used to protect wood, and are less toxic than many commercial termiticides.

Pathogens that affect higher animals are also controlled by the compositions of the present invention, including fungi and microbes such as trypanosomes and other protozoans or bacteria. The compositions can be used to kill or control the growth of microbes in infected organisms, in hosts or vectors that support and transmit such microbes, and in or on substances suspected of being contaminated with such microbes. The compositions may be administered or delivered by conventional means. For administration to a subject such as a human, a composition of about 30 brix concentration is prepared from a culture of L. sulphureus or G. lucidum by methods described herein. It may be diluted, typically by 10× to 100×, and can then be administered orally or parenterally to the subject; the route of administration depends upon the type of pathogen to be treated, and selection of such methods is within the ordinary skill in the art. A single dose may be beneficial, but typically the subject will be dosed at least twice, and frequently the subject will be treated with at least one dose per day for 2-7 days.

For application to surfaces or substances in need of pathogen control, a composition as described above is prepared, and it is then diluted by a 1:100 to 1:2,000 ratio, after which it can be applied directly to the surface or substance to be treated, using conventional means.

The compositions may also be used to treat or control fungal and parasitic infections such as ringworm, tapeworms, and the like. These treatments may be done by dermal administration for pathogens such as ringworm, skin infections, toenail fungi, athlete's foot and the like: the composition may be applied directly to the affected area, optionally accompanied by a carrier such as DMSO that facilitates transdermal delivery of compounds that otherwise penetrate the skin slowly. For these applications, it is sometimes desirable to concentrate a composition of the invention, such as one prepared from a culture of L. sulphureus or G. lucidum, to dryness or nearly to dryness, and to dissolve or suspend a sample in a solvent or solvent mixture that is acceptable for dermal applications, such as a solution in ethanol or isopropyl alcohol and water, or in DMSO. Applications may be repeated until a curative effect is observed.

Because of the low cost and low toxicity of these compositions, they may be useful for the treatment of exposed surfaces; water, including drinking water supplies, surface waters, and wells; and other surfaces or areas that may become contaminated. Their application can reduce the exposure of animals and humans to infectious pathogens on such surfaces or in such substances.

The antipathogenic activity of the compositions may also be used to retard growth of or to kill bacteria, nematodes, protozoans and the like, which cause diseases in mammals, including humans. The compositions have been shown to have activity against protozoans that cause human diseases, including malaria. The compositions can thus be used to treat such diseases in animals and in humans, and to deter transmission of such diseases by killing or stopping growth of the pathogens outside a host's body, or by administering the compositions to host or vector organisms that facilitate transmission of the pathogen. The compositions can be used in partially purified form for treatment or prevention of such diseases, and may be administered by conventional means.

Preparation of compositions for use in treating animals and/or humans may include an additional step of purification such as ultrafiltration to ensure sterility, and may include added components such as stabilizers and/or preservatives to maintain them in a suitable form for medical uses.

The selection of a growing medium that enhances production of the bioactive compositions of the invention is another aspect of the invention. Culture fluids of G. lucidum and L. sulphureus have nematicidal activity, for example. This activity can be increased by cultivation in a suitable rich medium. For example, when PDB (potato dextrose broth, a conventional growing medium) was used to produce the bioactive culture fluids, the nematicidal activity was significantly lower than that observed from culture fluids produced in a special Rich Broth Medium (RBM). This enhancement of bioactivity by using RBM as the growing medium was observed in the culture fluids produced by both G. lucidum and L. sulphureus. RBM is a rich medium that contains different carbon sources, including corn gluten, molasses, oat meal, brewer's yeast, vegetable oil, sucrose, etc. whereas PDB contains potato starch as the main source of carbon.

The invention provides methods to produce biocidal oligosaccharides or compositions containing them, where the methods comprise growing a Basidiomycete fungus in a substantially liquid medium. The medium used has a significant effect on the amount of the bioactive oligosaccharides produced; thus the constitution of the growth medium is another aspect of the invention, and careful selection of the growing medium can be used to modify the metabolite content and thus the biological activity of the compositions. Methods to concentrate and/or partially purify active forms of the compositions of the invention are also provided, including methods to concentrate the actives without excessive heating in order to preserve the bioactivity. The compositions of the invention are useful for application to living plants or the locus where such plants are grown, to reduce the growth of plant pathogens. They may also be used to treat or supplement the medium in which a plant to be protected is grown, such as by being admixed with a hydroponic medium for growing plants substantially without soil. The compositions are also useful to reduce the adverse effects that pathogens have on infected plants and plant products, even where they do not eliminate the pathogen; they appear to operate by systemically activating the plant's natural defenses to promote pathogen resistance or injury resistance. Thus another aspect of the invention includes methods to use these compounds and compositions to reduce adverse effects of pathogens on plants and plant products.

### BRIEF DESCRIPTION OF THE DRAWINGS

FIG. 1 shows a GC-mass spectrum trace of an active component from a G. lucidum culture fluid that has been silanated for analysis.

FIG. 2 shows a mass spectrum of one peak from the GC-mass spectrum in FIG. 1.

FIG. 3 shows a mass spectrum of one peak from the GC-mass spectrum in FIG. 1.

FIG. 4 shows a mass spectrum of one peak from the GC-mass spectrum in FIG. 1.

FIG. 5 shows the pH dependence of the growth rate of G. lucidum and L. sulphureus.

FIG. 6 shows the temperature dependence of the growth rate of G. lucidum and L. sulphureus.

### MODES OF CARRYING OUT THE INVENTION

As used herein, the terms “carbohydrate” and “oligosaccharide” are used to describe any compound that is at least 50% by weight composed of monosaccharides or derivatives of monosaccharides. “Oligosaccharides” contain a linear or branched chain of at least five monosaccharides or derivatives thereof, while carbohydrates include mono- and di-saccharides and smaller chains of monosaccharides as well as derivatives of these as well as oligosaccharides.

The type of linkage between the monosaccharides is not important as long as each linkage comprises an ether group. Derivatives of monosaccharides include aminosugars, O-acyl sugars, deoxy-sugars and the like. A “carbohydrate” or “oligosaccharide” may include other chemical structural features in addition to the chain of monosaccharides or derivatives thereof. “Carbohydrates” as used herein are primarily composed of monosaccharides, i.e., at least 50% of the compound's molecular weight is from monosaccharide groups; the monosaccharide groups or derivatives thereof in a carbohydrate may be but need not be in a straight chain or branched chain.

The antibacterial compositions comprise at least one bioactive compound that is stable to proteinase activity, and that comprises an oligosaccharide structure and has a total molecular weight between about 2,000 and 5,000. The compositions are at least partially purified products produced by growing a Basidiomycete fungus, and are useful because they possess biocidal activity against at least one type of plant pathogen selected from bacteria, fungi and nematodes, or against at least one pathogen that infects higher animals, including bacteria, fungi, nematodes, and various parasites.

Compounds and compositions of the invention are “active” if they provide at least 50% growth reduction of a particular species of interest or if they provide a statistically significant protective effect of at least one cultivated plant or plant product. Thus compositions are active as plant bactericidal agents if they are active against at least one species of phytopathogenic bacterium, for example.

Compounds and compositions are “biocidal” as used herein if they are active against at least one species of plant pathogenic bacterium, fungus or nematode.

In one aspect, the present invention provides certain compounds and compositions that are useful to protect plants, plant products, and fungi from the adverse effects caused by various phytopathogens. The compositions of the invention comprise biocidal oligosaccharides that are produced by Basidiomycete fungi. The oligosaccharide compounds are partially characterized by structure, and are more fully defined by their physical properties, stability, and bioactivity, especially their biocidal activities against plant pathogenic bacteria, fungi and nematodes, and by their method of production.

In another aspect, the invention provides methods of producing certain bioactive compounds and compositions using at least one Basidiomycete fungus. The methods comprise growing a fungus in a substantially liquid medium that comprises carbon sources and other nutrients to support the growth of the fungus. The fungus excretes at least one bioactive compounds into the culture fluid in which the fungus is growing. Thus the invention provides methods of growing a Basidiomycete fungus that are conducive to the accumulation of at least one biocidal metabolite in the culture fluid, from which a composition of the invention can be prepared.

In another aspect, the invention provides methods of using these bioactive compounds and compositions to reduce the adverse effects of various pathogens on plants, plant products, or fungi such as cultivated row crops, ornamentals, trees and vines, and mushrooms. The methods comprise contacting a plant, plant product, or fungus to be protected or its locus or its growing medium with a compound or composition as described more fully herein.

The invention also provides methods for processing a culture fluid to produce a tea or other refined composition having useful concentrations of the bioactive species described herein. These methods include growing the fungi on a medium as described herein, which in some embodiments comprises at least about 10% fruit juices, and using a growing chamber that provides, in addition to the surfaces of the vessel itself, additional mycelium-supporting growing surfaces that are immersed in or in contact with the growing medium. These surfaces encourage more and faster mycelial growth, which enhances the production of the active components of the compositions; thus it is possible to grow the fungal culture more rapidly and produce a greater quantity of the active compositions with a given culture volume. Typically, the total growing surfaces, including the container, provide at least about 50 cm² per liter of medium, and preferably at least 100 cm² per liter of medium, or at least about 200 cm² per liter of medium, and frequently the volume is at least about 100 liters per container. Ideally, the additional surfaces provide at least a 50% increase in available surface area per liter of medium relative to the container alone. Using these methods, the culture produces more of the active oligosaccharide compositions, so a given volume of medium produces a composition having greater activity. In some such methods, the broth is mixed or agitated without substantially disturbing the mycelial mat during the growing phase. While the production methods generally are not complex, the improved method increases productivity and better utilizes the space and time required to produce compositions as described herein.

The preferred fungi for producing the compounds and compositions of the invention are Basidiomycetes, a group of fungi that coexist with, and depend for growth on, plants. Basidiomycetes assume many different forms: they include species that live on dead plant materials including puffballs and some ‘classic’ mushrooms, some of which are edible and some of which are quite toxic; they also include microscopic fungi that are referred to as rusts or smuts, which are the cause of important economic damage to various crop plants and plant products. Laetiporus species of Basidiomycetes are preferred for some embodiments of the invention, and are particularly useful for producing compositions having antibacterial activity, including activity against protozoans that cause malaria, leishmaniasis, and other diseases; trypanososomes that cause Chagas disease, nematodes that cause filariasis, elephantitis, and canine heartworms, and the like; and other pathogens that affect humans and domestic animals. L. sulphureus is sometimes preferred because of its favorable growth habits. Ganoderma species are preferred in some embodiments, and G. lucidum is sometimes preferred.

While some Basidiomycete fungi grow on decaying residues from dead plants, it has now been found that Basidiomycete fungi, especially those grown in a substantially liquid culture, produce compounds that are useful to protect growing plants from the adverse effects of many pathogens, including bacteria, fungi, and nematodes.

A preferred Basidiomycete genus for producing the compounds and compositions of the invention is the genus Ganoderma. In particular, Ganoderma lucidum, a species that grows on decaying tree residues, has been shown to produce significant amounts of the bioactive substances of the invention when cultured in an organic-enriched aqueous medium. Moreover, an active composition can be prepared from the culture fluid on which G. lucidum is grown, and the titer of the active components in the culture fluid can be increased by use of suitable growing conditions as described herein.

Another preferred Basidiomycete genus for use in the invention is Laetiporus, especially Laetiporus sulphureus. Laetiporus sulphureus “Sulphur shelf” or “Chicken of the Woods” is a wound parasite of hardwood trees. It is commonly found in Hawaii on Eucalyptus robusta. It, too, can be grown conveniently in liquid culture to produce the compositions of the invention.

Members of other genera in the Basidiomycete family may also be used to produce bioactive oligosaccharides within the scope of the invention. These include:

Agaricales
Agaricaceae
Bolbitiaceae
Clavariaceae
Coprinaceae
Cortinariaceae
Entolomataceae
Fistulinaceae
Hydnangiaceae
Lycoperdaceae
Marasmiaceae
Nidulariaceae
Pleurotaceae
Pluteaceae
Strophariaceae
Tricholomataceae

Boletales
Boletaceae
Gyroporaceae
Paxillaceae
Sclerodermataceae
Suillaceae

Cantharellales
Cantharellaceae
Clavulinaceae
Hydnaceae

Dacrymycetales
Dacrymycetaceae

Exobasidiales
Exobasidiaceae

Hymenochaetales
Hymenochaetaceae
Schizoporaceae

Phallales
Gomphaceae
Phallaceae
Ramariaceae

Polyporales
Fomitopsidaceae
Ganodermataceae
Gloeophyllaceae
Hapalopilaceae
Meripilaceae
Meruliaceae
Phanerochaetaceae
Podoscyphaceae
Polyporaceae
Steccherinaceae

Russulales
Auriscalpiaceae
Bondarzewiaceae
Hericiaceae
Lachnocladiaceae
Russulaceae
Stereaceae

Thelephorales
Bankeraceae
Thelephoraceae

Tremellales
Exidiaceae
Tremellaceae

The bioactive compounds and compositions of the invention are produced by growing at least one Basidiomycete fungus under any of a wide variety of culture conditions. Some suitable culture conditions for growing Basidiomycetes are known in the art, and preferred conditions for use with at least certain species are described herein. Optimization of growing conditions for a particular species and bioactivity are within the ordinary skill in the art, using assay methods such as those described herein to select a medium with appropriate properties for the particular activity of interest.

The compositions may be produced under any conditions where the fungi grow at a reasonable rate. However, some media components and growing conditions have been shown to enhance production of bioactive oligosaccharides and other bioactive species. Optimum growth conditions for mycelia production were determined for G. lucidum and L. sulphureus under stationary conditions. It was observed that the highest mycelium concentration was produced in 2000 ml Erlenmeyer flasks, pH value of 3.0, and temperature of 30° C. These results differed from those reported by Yang et al., (1998) where they found optimal conditions for cultivating G. lucidum under shaking conditions were at pH 4.0, and temperature 35° C. Yang & Liau, “Effect of cultivating conditions on mycelial growth of Ganoderma lucidum in submerged flask cultures”, Bio. Eng. 19, 233-36 (1998). The cultivating conditions set forth herein for growth of these fungi were very effective for the production of antimicrobial compounds.

Culture fluids from G. lucidum and L. sulphureus were also observed to have fungicidal activity against most phytopathogenic fungi tested. This activity is stable in aqueous solution at ambient temperatures but is heat labile, suggesting that this activity is of a different nature than the bactericidal activity and is likely due to a different chemical species in the crude culture fluid. A few reports have shown that some species of mushrooms have fungicidal activity on several plant pathogenic fungi. However, the fungicidal activity of G. lucidum has not been reported previously against the pathogens used herein, and this is believed to be the first report of such activity with L. sulphureus.

Culture fluids of G. lucidum and L. sulphureus have nematicidal activity, also. This activity can be increased by cultivation in rich medium. For example, when PDB was used to produce the bioactive culture fluids, the nematicidal activity was significantly lower than that observed by culture fluids produced in RBM. RBM is a rich medium that contains different carbon sources, including corn gluten, molasses, oat meal, brewer's yeast, vegetable oil, sucrose, etc. whereas PDB contains potato starch as the main source of carbon.

The compositions of the invention are also active against pathogens that infect animals, including humans, including activity against protozoans that cause malaria, leishmaniasis, and other diseases; trypanososomes that cause Chagas disease, nematodes that cause filariasis, elephantitis, and canine heartworms, and the like; and other pathogens that affect humans and domestic animals, including anthrax, yellow fever, dengue fever, Japanese encephalitis, smallpox, cholera, leishmaniasis, and tuberculosis. For example, culture fluids of Laetiporus sulphureus were found to kill about half of exposed malaria protozoans when used as a 1:2000 dilution of a 30 brix syrup. The 30 brix syrup was produced by a ‘short boil’ process, where the composition, after removing insoluble materials, was heated at boiling for a few seconds only and was then simmered at about 170° F. until the 30 brix concentration was reached. Methods for administering these compositions to deliver antipathogenic activity in vivo are known in the art, and include conventional means such as oral delivery of a liquid or solid form of a composition of the invention, intravenous delivery such as by IV, and injection of a concentrated solution parenterally or intramuscularly. Suitable formulation methods for such compositions are known in the art, as described for example in Remington's Pharmaceutical Sciences, 18^{th} Ed.

In one aspect, the invention provides growing conditions and a liquid medium that enhance the production of the bioactive compounds of the invention. In some embodiments, the growing medium comprises plant-derived carbohydrates including processed plant materials such as oatmeal, sugars, potato, agar, and the like in an aqueous suspension. In some embodiments, the medium is supplemented with a vegetable oil such as corn oil, canola oil, or similar plant-derived oils. A particular combination of readily available and inexpensive materials has been found to enhance production of the biocidal compositions; it is referred to herein as Rich Broth Medium (RBM). RBM is typically produced by combining the following materials in water: oatmeal, brewer's yeast, corn gluten, molasses, citric acid, and canola oil. A preferred RBM mixture is prepared by mixing 15 g of ground oatmeal, 15 g brewer's yeast, 15 g corn gluten, 1 tsp molasses, 2 g citric acid, and 2 ml of canola oil per liter of water, and sterilizing it in an autoclave before inoculating it with a fungus. Other desirable components for the growing medium include sucrose, malt extract, yeast extract, potato infusion, agar, and the like. Fruit juices and residues of fruit growing and processing can also be used to provide additional growing material for the fungi, and manipulation of the fruit content can be used to optimize the activity of the composition.

Thus in one embodiment, the invention comprises a composition as described herein that is produced by growing a Basidiomycete fungal species on RBM or a substantially similar medium. Preferred Basidiomycete species for this use include G. lucidum and L. sulphureus. For some embodiments, the preferred Basidiomycetes exclude the Laetiporus species.

In another embodiment, the oligosaccharide-containing compositions are produced by growing a Basidiomycete fungal species on Potato Dextrose Broth, or a substantially similar medium. This medium comprises potato infusion and dextrose, and may be used at a pH of about 5.1 or at a pH optimal for the particular application, and is well known in the art.

Combinations of the components of these media, additional materials that may beneficially added to provide a balanced growing medium for a particular fungal species, and other similar nutrient sources will be apparent to the skilled artisan from the growing methods described herein, and use of media containing those substances to produce the bioactive compositions described herein are also within the scope of the invention.

In another aspect, the invention provides improved methods for producing a fungal culture fluid that increases the rate of growth of the fungus and the rate of production of the active substances in the culture fluid by increasing the ratio of growing surface area to medium volume. The improved methods are especially useful for large scale production, because they maximize the yield of the bioactive substances while reducing the growing space required, and can also shorten the growing cycle. The methods are especially useful for certain Basidiomycete species, including Laetiporus cultures.

Typically, for producing a large volume of the compositions of the invention, a culture is grown on an aqueous medium in a barrel, drum, vat, or similar container for about 30 days before a substantial amount of bioactive substance is present in the culture fluid; maximum production of the bioactive species may require maintaining the culture for another 30-60 days. It has now been found that providing additional growing surface area accelerates the rate of growth of the fungus and the rate of bioactive substance production in a given volume of medium. It can also shorten the cycle time for growing batches of culture fluids using the culture methods described herein.

Growing surface area refers to any surface that can support mycelial growth or provide an attachment for mycelia. Providing additional growing surface refers to providing any surfaces other than the surfaces naturally associated with the container itself, as long as the additional surfaces are in contact with or proximal to the medium such that mycelial growth readily occurs on the added surfaces.

The growing container for a fungal culture used to produce large quantities of the compositions of the invention is typically a barrel or vat or similar container, made of a material that is suitable for holding an aqueous medium containing materials essential for fungal growth. Various plastics, glass, PLEXIGLAS™, fiberglass, and certain metals are suitable materials for such containers. Typically, however, these containers provide a relatively low surface area to volume ratio when the medium depth is more than a few inches, and it has been found that the rate of growth and of production of the bioactive species of the compositions of the invention increase when the surface area to volume ratio increases. For large scale production, it is preferable to increase the depth of the medium as much as possible in order to maximize the utilization of growing space and light, while producing as much of the culture fluid as possible in each batch. Many fungal cultures suitable for producing the compositions of the invention can be grown efficiently with medium depths much greater than a few inches, and of course the surface area to volume ratio usually drops as the medium depth is increased. Often it is beneficial to provide room for more extended mycelia mats and increasing the surface area available to support mycelial mat growth. Thus it has now been demonstrated that the growth rate of the culture and the rate of production of the bioactive species of the invention both increase when medium depth exceeds a few inches if additional growing surfaces are provided.

In certain embodiments, the invention thus provides additional growing surfaces that are typically substantially vertical and that extend from at or above the surface of the growing medium through the surface of the growing medium and down into the medium, at least part of the way to the bottom of the container in which the fungal culture is grown. In some embodiments, the additional surface area is provided by suspending components from above the surface of the medium so that they hang down into the medium; in others, the additional surface area is provided by surfaces that float or are supported by material that floats on top of the medium. In other embodiments, the structure(s) providing additional growing surfaces extend from the sides or from the bottom of the container into the medium, and usually they extend through the surface of the medium and upwards above the medium to provide additional growing surface that is above the medium but in fluid contact with the medium. The most benefit is obtained from structures that extend upward from the surface of the medium, so that the additional growing surface is in contact with the medium to remain most either directly from the medium or from mycelia that reach into the medium and is also exposed to air.

Often, the additional growing surfaces are in the shape of rods, flat plates, strips, tubes, or cylinders; they may also be provided by fin-like projections that extend from the sides or bottom of the container or both. In one embodiment, the additional growing surfaces are provided by a plurality of plates of e.g. PLEXIGLAS™ that are suspended from a lid that is used to cover a drum or barrel or other container in which the fungal culture is grown. Optionally, the plates may be interconnected such as in a checkerboard pattern, and there may be at least one corresponding structure extending from the bottom or side of the container to stabilize the additional growing surface structures when they are so suspended, providing improved stability. Having the additional growing surfaces held relatively stationary is beneficial to the growing fungi, since movement of the growing support can damage the fungus once it is established. While not required to realize some of the advantages of the improved methods, having the additional growing surfaces remain relatively stationary throughout most of the growing phase is often an advantage. However, the shape of the additional growing surface and how it is supported or held in place is unimportant, as long as it provides surface area to which growing mycelia can adhere.

The additional growing surface is constructed of material that is suitable to support fungal growth above and/or below the medium surface. It may comprise one or more such materials, and may be of the same material as the container or of a different compatible material. Optionally, the material used for the additional growing surfaces may be sanded, scratched, scraped or otherwise roughened to encourage the fungus to adhere to and ‘climb up’ the additional growing surfaces. In some embodiments where the surface is otherwise a relatively smooth solid like glass or PLEXIGLAS™, it is advantageous to apply vertical scores, grooves or scratches: these encourage upward growth of fungus away from the medium, and may provide a degree of capillary action to encourage moisture to travel upward from the medium, further encouraging fungal growth. Instead of a solid surface, the additional growing surface can also comprise a porous or absorbent material such as a cloth, sponge, or mat that may be composed of a plastic or fiberglass, for example; or it may be in the form of a perforated plate or a mesh or screen.

Preferred materials for the additional growing surfaces are those suitable for long-term exposure to an aqueous growing medium useful for supporting fungal growth; typically this includes the same materials used for construction of the containers in which the cultures are grown. Stainless steel, polyethylene, polypropylene, polystyrene, nylon, polyvinyl chloride, fiberglass, polyurethane, and TEFLON™ can all be used, for example. Stainless steel mesh or screen works well, as do polypropylene mats, cured polyurethane foam such as acoustic material, and TEFLON™. Each of these is sometimes a preferred material. Combinations of these materials and of their shapes and textures may be employed, and different methods for holding them in place can be combined as well.

Another aspect of the invention provides a compost tea produced by fungal digestion of plant-derived organic materials. The compost tea is produced by composting an aqueous suspension of plant materials with a Basidiomycete fungus for several weeks or longer, typically, and partially purifying the product to provide typically a solution of the active compounds that is substantially free of cellular derived materials. Importantly, to provide certain of the benefits by application of the composition to foliage or fruit, the compositions are prepared without heating the material during processing to a temperature above about 50° C. or above 70° C. or above 90° C., since such heating reduces or destroys some of the bioactive species that provide protection from fungal and/or nematicidal injury. These plant protective compositions can be applied to a plant's foliage, but they can also be applied directly to certain crops, including e.g. onions and papayas, to reduce fungal injury to the fruit or vegetable to be consumed, thus improving product appearance and quality.

Thus in one embodiment, a mixture of plant products comprising by-products from production and processing of pineapple, papaya, and sugarcane combined with processed plant materials such as oatmeal, wheat gluten, and baker's yeast, is digested for 2-5 weeks in an aqueous suspension/slurry under conditions such as those described herein. The compost tea is prepared for use without heating, which would destroy some of the desired bioactivities. The solids are separated by conventional methods, and optionally the tea is further purified by removal of high molecular weight and cellular derived materials by conventional methods such as ultrafiltration. This produces a liquid that can optionally be further purified, or it can be used directly on the foliage of a plant, such as a tomato plant, or to a plant product, such as a growing papaya, to reduce fungal injury to the plant or plant product.

Other aspects of the invention provide culture conditions for growing the fungus to produce a culture fluid rich in the desired biologically active components. Typically it is advantageous in producing the compounds and compositions of the invention to grow a Basidiomycete on a substantially liquid medium at a pH that is below 7, and preferably below 5 in order to enhance production of the biocidal oligosaccharides. In some embodiments, the liquid medium is maintained at a pH below 4 during the majority of the time or preferably all of the time that the culture is maintained until the culture fluid is collected. In other embodiments it is maintained at a pH below 3.5; in a preferred embodiment the pH of the medium is maintained at a pH of about 3 throughout most of the culture growing period. The pH can be established using any biocompatible base such as sodium or potassium hydroxide, and is maintained using suitable buffers as needed. Optionally, a pH meter and automated system for maintaining the pH can be employed to prevent significant excursions away from the desired pH.

The temperature of the culture should also be maintained for optimal growth of the Basidiomycete fungus, and to maximize production of the desired bioactive components. While the optimum temperature will depend on the species of Basidiomycete being grown and on the precise medium used, in most embodiments a growing temperature of between about 20° C. and about 40° C. is preferred. In some embodiments the temperature is maintained between 25° C. and 35° C., preferably at about 30° C. or at about 30-35° C. during the majority of the culture growth phase. While temperature excursions somewhat above or below this range are not necessarily damaging to the culture fluid or the growing fungus, they may cause a reduction in yield of the desired bioactive components in the culture fluid, or they may make it necessary to prolong the growing phase. The temperature of the culture can be maintained using a thermostat and heating and cooling systems that are well known in the art.

The medium may be agitated by stirring it or bubbling gas through it, for example. In a particular embodiment, however, the medium was not agitated. Instead, it was maintained as a very shallow suspension, where the culture was not over about 1-6 inches deep, or about 2 inches, or about 3 inches, or about 4-5 inches deep: if a deeper suspension is used and the culture depth is significantly greater than 6 inches, some form of agitation or aeration may be desired.

For larger scale production, it is sometimes preferred to agitate the medium. Significant improvement in growth for large scale production is sometimes achieved when suitable agitation is used to mix the growing medium and to increase contact of the medium with the mycelia, without unduly disturbing the mycelial mats. This is beneficial because a stationary culture tends to leave some of the medium to remote to make contact with the fungus, and thus the medium not near the mycelia neither provides nutrient to the fungus nor receives exudates that include the bioactive chemical species of interest. This mixing can be achieved by gentle agitation of the medium in which the fungus grows or by bathing the fungal growth above the medium with the growing medium. In some embodiments it is accomplished by a lifting pump system that takes medium from near the bottom of the growing container, or at least from a point below the majority of the fungal growth at the particular growth stage, and distributes it over the growing fungus. The medium can be sprinkled, dripped or sprayed onto the growing fungal mat, or it can be allowed to flow gently enough over the growing fungus to avoid disruption of its growth. It can also simply be redirected into the container in a way that encourages mixing, e.g. it can be returned to the container at a point sufficiently removed from the point where it is taken in by the pumping system so that the net result is a gently current within the medium. Alternatively, a subsurface pumping system or mixing device such as a stirring mechanism can be used to gently direct fluid that is not in contact with the mycelia into or onto the mycelial mat from below, without unduly disturbing the mat structure. In this way, a relatively small growing culture can produce a large volume of culture fluid containing the compositions of the invention.

The amount of additional growing surface is not critical: any additional growing surface provides some benefit. However, it is often desirable to increase the available surface area by at least about 50% or by 100% or more. In some embodiments, for example, a 208 liter drum is used to contain a culture, and it is charged with about 113 liters of medium. In a vertical orientation, i.e. when standing upright, it has a surface area/volume ratio of about 22 cm²/liter. When placed on its side, that ratio increases to about 42 cm²/liter. However, addition of a few plates of additional growing surface material as described herein can easily double or triple the available surface area; and the use of a porous material may provide even greater increases in surface area. In some embodiments, it is preferable to provide additional growing surfaces to bring the surface area/volume ratio to above 50 cm²/liter, or above about 100 cm²/liter.

As different materials and shapes of such additional support surfaces and different fluid mixing or redistribution arrangements may be preferred depending upon the specific fungus used, the growing conditions and medium, and the depth of the medium, it may be necessary to select a suitable combination of these features when scaling up production of a particular culture. Methods provided herein for bioassays can be used to determine which conditions provide the greatest amount of a desire bioactive species. Thus with routine experimentation guided by the bioassays provided herein, these parameters can be optimized for each particular culture and/or growing environment.

The length of time for growing the Basidiomycete on the liquid medium to optimize the yield of the bioactive substances of interest depends on a variety of factors, including the medium, pH, temperature, and fungal species employed. The precise time is not critical to the successful generation of the bioactive components, as they are substantially stable under the culture conditions once produced. Typically the growing phase will last at least several days to a week; in some embodiments it is about two weeks; and in some it is advantageous to maintain the culture for about three weeks or about four weeks or up to about 60 days. In some embodiments the culture is maintained at least five weeks, and in some embodiments it is maintained six weeks or longer. Once the growing phase has been completed, the culture fluid may be harvested, though it is not critical to harvest the fluid immediately.

Thus in one embodiment, a Basidiomycete fungus may be grown at a temperature of about 30° C. and a pH of about 3, in RBM, without aeration or agitation, preferably in a shallow mixture less than about 6″ deep. In another embodiment, a Basidiomycete fungus may be grown at a temperature of about 30° C. and a pH of about 3, in RBM or a similar medium in barrels, drums or vats having additional growing surfaces provided as described herein and optionally using an agitation method such as those described above.

The culture fluid is harvested by draining or decanting it from the growing mycelial mat before use, or by removing the majority of the fungal growth by mechanical separation means such as filtration or centrifugation. Typically, the harvested culture fluid is at least partially purified before use. In some embodiments, solids are removed by e.g., sedimentation, filtration, or centrifugation or some combination of these. Optionally, the solution may also be sterilized by known methods such as heating and/or filtration or ultrafiltration to provide an aqueous solution containing at least one biologically active oligosaccharide compound of the invention as an aqueous solution, which may be sterile. Preferably, sterilization is done by heating the aqueous solution at a temperature of up to about 100° C., or by filtration with a membrane such as a 0.22 micron membrane, or by UV or gamma irradiation.

Effective sterilization may also be achieved by lyophilization or by a reverse osmosis process. Combinations of isolation and purification methods may also be employed to provide other compositions that are at least partially purified relative to the crude broth. Further purification of the culture fluid or of the bioactive compounds therein may also be undertaken as desired, using methods known in the art based on the information provided herein about the structure and stability of the active compounds. This provides a partially purified composition that is novel and has useful biological activity for the protection of plants against damage caused by pathogens.

The concentration and handling properties of the compositions when in liquid form may be adjusted using methods known in the art for concentration, adjusting density, surface tension, viscosity, and other mechanical properties. Frequently, the processing of a culture broth includes at least one heat-based concentration step to reduce the volume of the composition and increase its concentration of active materials. In some such embodiments, the composition is heated to boiling for a limited period of time, preferably not over about 4 hours, and in some embodiments it is boiled for an hour or less to preserve the biological activity of the composition. Further concentration is accomplished by heating at a lower temperature, or by other conventional methods. For some uses, the composition is concentration to a syrup having a concentration of at least 10 brix, or at least 20 brix, or at least about 30 brix, without heating at a temperature above 90° C. for more than one hour.

The oligosaccharide-containing compositions of the invention may comprise a number of different chemical species, and the active components are not yet fully characterized by chemical structure. The antibacterial compounds are soluble in water or methanol, but not significantly soluble in dichloromethane or ethyl acetate. By HPLC, using an ACPI detector (chemical ionization), the compounds show an apparent molecular weight of 3000 to 4500. The methanol soluble material comprises several components, but the antibacterial activity is in the fast-eluting, highly polar fractions. Mass spectral analysis of the active components showed fragmentation patterns characteristic of an oligosaccharide: successive losses of 18 units in molecular weight, corresponding to losses of H₂O were observed.

According to a gel filtration column the antibacterial compounds fall within a defined molecular weight range of about 4000-4500. The structures of the compounds are thus substantially defined by their molecular weight and by the presence in the structures of certain monosaccharide components, which make up most of the structure, as well as by their solubility, stability, and bioactivity characteristics.

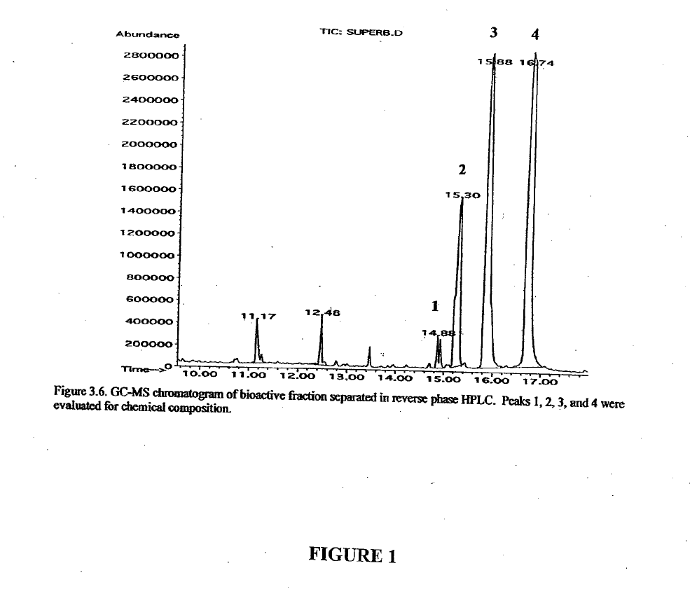

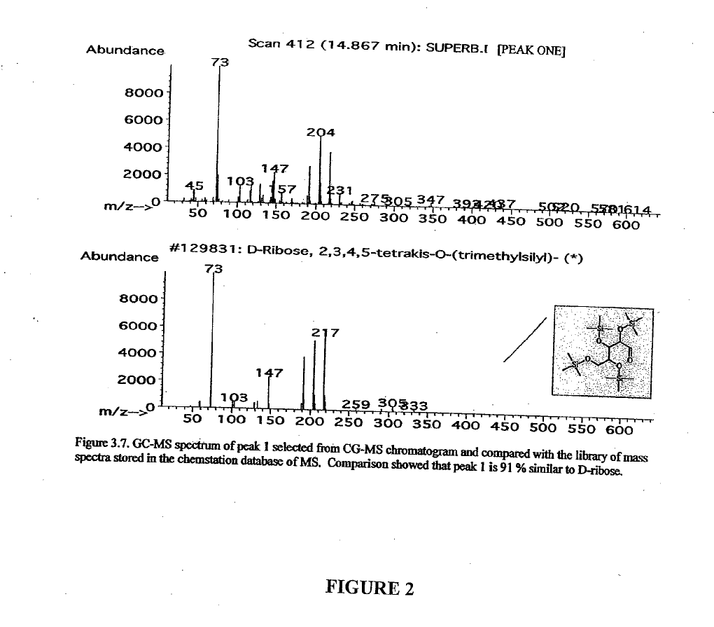

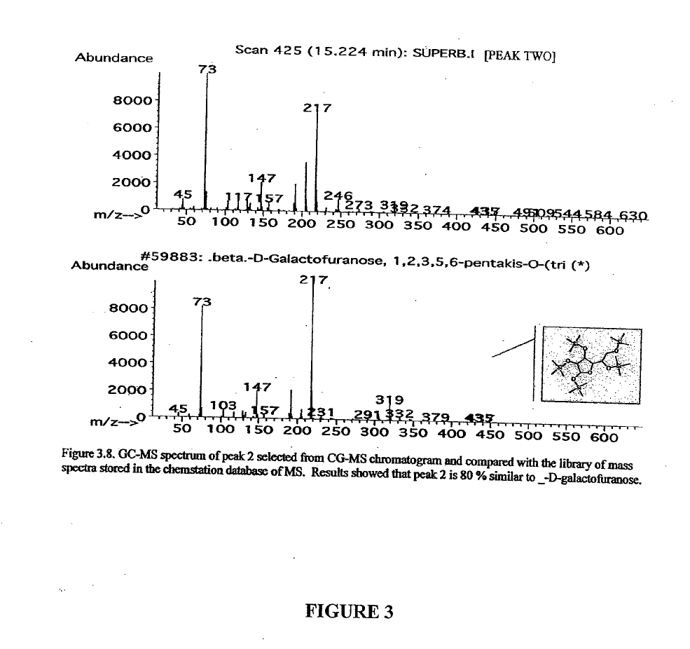

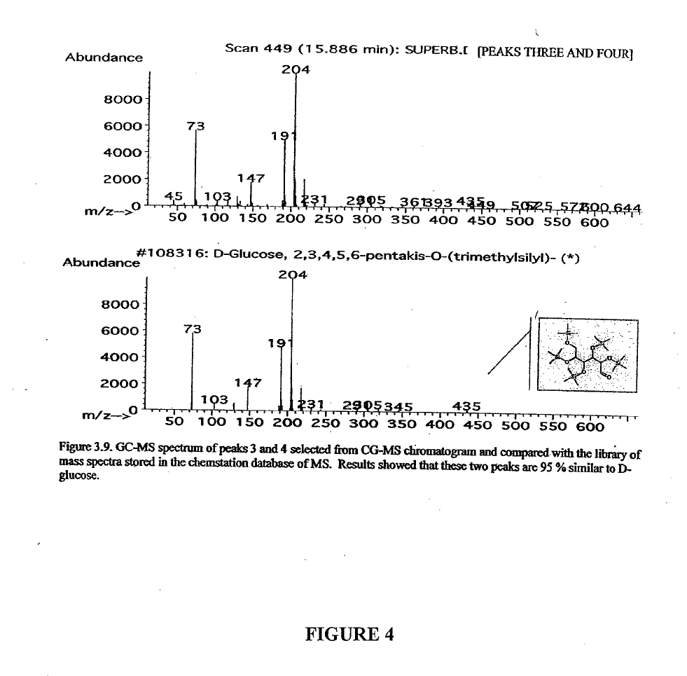

While the presence of other structural features has not been ruled out, the antibacterial compounds of the invention are primarily oligosaccharide structures composed of a few common monosaccharides. This was demonstrated by mass spectral analysis of samples of the active compounds that were silanated with trimethylsilyl chloride. The GC-MS shows only monomers, since the parent compounds are not volatile enough to show up; it provided a mass spectrum for each of four peaks. These were compared to a database of mass spectral data, and indicated that ribose, galactofuranose and glucose were present in the oligosaccharide. See FIGS. 1-4. Ribose and glucose each appeared as two separate peaks, which may be attributable to seeing them as a mixture of anomers. Galactofuranose is a sugar that is not naturally present in animals, but is broadly distributed in pathogenic organisms (see Beverley, et al., Eukaryotic Cell, 1147-54 (June 2005)); thus, without limiting the invention be any theory of operation, its presence in the pathogenic compositions may relate to their mode of action.

The antibacterial active substance(s) present appear to contain no peptide linkage that is essential to their activity: they exhibited no loss of antibacterial activity when treated with a known protease, Proteinase K. The antibacterial actives also appear to be stable to heating in aqueous medium: boiling a sample in water at 100° C. for an hour, or storing it at room temperature for at least 90 days, did not substantially reduce its antibacterial activity. Heating in water as described did, however, significantly reduce the antifungal and nematicidal activities of the compositions. Therefore, the compositions appear to comprise at least two different active compounds; furthermore, the antibacterial activity of the compositions can readily be separated from the nematicidal and fungicidal activities by heating an aqueous solution of the composition to remove the latter activities.

The antibacterial compounds of the invention are believed to contain oligomers of common monosaccharides. These are all presumed to be the D-isomers, though the GC-MS data cannot confirm absolute stereochemistry. The ratio of these cannot be accurately determined from the GC data, but the data suggest that an active oligosaccharide comprises one or possibly two ribose units, several galactose units, and most of the balance is glucose. The active antibacterial substances are therefore believed to be oligosaccharides comprising primarily these common monosaccharide moieties.

The compositions of the invention are typically purified at least enough to be substantially free of cellular components. This means that any fungal-derived insoluble material has been substantially removed from the aqueous culture fluid, as by the purification methods described herein. In some embodiments, the culture fluid is used after it has been filtered or otherwise treated (e.g. by sedimentation, centrifugation, dialysis, etc.) to remove substantially all of the suspended solids present, including the fungal-derived insoluble materials, and to remove cells and cellular debris over about 5 microns in size, providing an aqueous solution that is enriched in the biologically active compounds of the invention. In some embodiments, the composition is treated to remove components larger than about 1 micron in size.

Optionally, the solution is treated by ultrafiltration or by a gel chromatography, dialysis or other conventional process to remove substantially all materials that are above a certain size, such as the size of a cell, or such as an approximate molecular weight of about 100,000, or about 60,000, or about 40,000, or about 20,000, or about 10,000. These methods remove impurities from the biologically active compounds of interest, and they provide partially purified compositions that are novel and biologically active and that can be further purified or concentrated or can be formulated for use as pesticidal compositions. Removal of high molecular weight materials improves the aqueous solutions to be used for foliar applications by reducing the amount of undesired or unneeded material applied to plants and fruits.

These compositions maybe further purified by, for example, extraction with a water-immiscible organic solvent to remove lipophilic and/or colored materials, or by standard chromatographic methods including gel, normal phase, and reverse phase chromatography to reduce amounts of inactive or undesired material that would otherwise be applied to the treated vegetation. They may also be partially purified by other conventional methods such as decolorization using charcoal or other adsorbents that remove impurities but do not significantly remove the desired bioactive compounds. “Decolorization” as used herein, refers to the removal from an aqueous solution or suspension of at least enough of a dissolved or suspended colored material to significantly lighten or change the color of the aqueous solution. Decolorization provides a material that is better for foliar application because it reduces or eliminates staining of the treated foliage or fruit, which improves foliar absorption of light and improves the appearance and quality of fruit.

The term “lipophilic materials” as used herein refers to materials that preferentially distribute into a water-immiscible solvent, permitting them to be partially or substantially removed by extracting them from an aqueous solution using such water-immiscible solvent. Examples of lipophilic materials are compounds that are uncharged at the pH of the aqueous solution being purified and that have a log P greater than about 2 or greater than about 3 at that pH. “Log P” refers to the negative of the logarithm of an octanol/water partition coefficient for a molecule, and is a well-known parameter for evaluating lipophilicity. Methods for measuring or calculating log P values are well known, and methods for such aqueous/organic extractions to remove lipophilic substances from aqueous solutions are also well-known. Removal of lipophilic materials improves the aqueous solutions to be used for foliar applications by reducing the amount of undesired or unneeded material applied to plants and fruits.

The antibacterial compounds of the invention are distinguished from other products produced by Basidiomycete fungi by their chemical and biological properties. They do not appear to correspond to any known bioactives produced by Basidiomycetes based on their molecular weight and solubility and stability properties. First, they are preferentially soluble in water, having very little solubility in organic solvents other than methanol: when a one-gram sample of freeze-dried culture fluid was shaken in 2 mL of acetonitrile, chloroform or ethyl acetate for two hours, the soluble materials in the organic solvent showed no antibacterial activity. Thus the active compounds appear not to be terpenoids or other substantially organic-like metabolites. Second, their activity was shown to be unaffected by treatment with Proteinase K under conditions where peptides were digested. Thus the bioactive compounds appear to contain no proteinase-susceptible linkages—they are not essentially proteins or simple polypeptides. Third, they were shown to have a molecular weight between about 2000 and 5000. This distinguishes them from beta-glucans produced by some fungi that have immuno-modulating activities, but have molecular weights that are far above 5000.

The antibacterial compounds of the invention appear to consist largely of oligosaccharide, and to have a molecular weight between about 4000 and 4500 based on size exclusion chromatography. However, when a sample of the active composition from G. lucidum was placed in a dialysis bag having a molecular weight cut-off of about 3500, the active material dialyzed out of the bag and was found in the external solution. This indicates an apparent molecular weight of at most about 3500. An HPLC method using a mass spectral detection and chemical ionization to minimize fragmentation suggests that the molecular weight of the active species is between 3000 and 4500. As discussed above, there are apparently at least two bioactive compounds in the compositions, and the antibacterial activity at least is associated with one or more carbohydrate chemical species having a molecular weight in the 2000 to 5000 range, and probably in the 3000-4500 molecular weight range.

In another aspect, the invention provides a composition produced by the process of growing a Basidiomycete fungus in a medium that supports its growth, followed by isolation of the composition as an aqueous solution or suspension comprising the culture fluid. The compositions of the invention comprise at least one active compound that has the structural and stability properties described above, and can be at least partially purified by steps such as filtration to remove some or substantially all particulates; extractions to remove some or substantially all of the materials that are significantly soluble in organic solvents; dialysis, size exclusion chromatography, or similar methods to remove materials of substantially different molecular weight; ion exchange resin treatment to remove acidic and/or basic components; and treatment with chemical agents such as oxidizing agents, reducing agents, chelating agents, and the like to remove other undesired components. Optionally, the broth may be heated to eliminate its antifungal and/or nematicidal activities, if only the antibacterial activity is desired, and may then be partially purified by conventional methods including those mentioned herein.

Where necessary during the process of partial purification, the bioactive compounds of the invention can be located using bioassay techniques known in the art to track where the biological activity of interest resides. Thus, for example, an antibacterial composition of the invention can be prepared using various combinations of conventional purification methods, while tracking the active components by assaying for antibacterial activity against at least one plant-dwelling bacterium such as Agrobacterium tumefaciens, Agrobacterium rhizogenes, Acidovorax avenae, Brenneria quercina, Erwinia carotovora, Pantoea herbicola, Pseudomonas corrugate, Pseudomonas syringae, Raythayibacter tritici, Xanghomonas axonopodis, or Xanthomonas campestris. Certain compositions of the invention are active against each of these pathogens. Purification can be undertaken to achieve any desired level of purity up to and including isolation of the active compounds in substantially pure form. Suitable methods for this isolation process are well known, and assay procedures are outlined herein to permit the user to locate antibacterial, antifungal, and nematicidal active species. Using such purification methods beyond simply heating and filtering produces a partially purified composition that is novel and is useful for antibacterial treatment of plants or plant products, or for reducing damage caused by bacteria and (if not heated) other plant pathogens.

The partially purified material produced by the foregoing methods may be used directly for treatment of growing plants or a medium or location where plants will be grown or where seeds have been or will be introduced. Alternatively, at any stage of purification, the compositions can be concentrated by known methods such as distillation, evaporation, or dialysis to produce a concentrated solution, emulsion, suspension, paste, or solid. The partially purified material, or a concentrated form thereof, can then be applied using conventional methods in an amount sufficient to reduce a detrimental effect caused by a plant pathogen, which may be a bacterium, fungus or nematode, or to slow or prevent the growth of such pathogens or the damage caused by such pathogens.

The compositions of the invention are useful in the cultivation of plants and fungi because they reduce the adverse effects of pathogens thereon. They can be administered to reduce the adverse effects of microorganisms, fungi, or nematodes by killing such pathogens, or by slowing the growth of such pathogens. The compounds and compositions can be deployed using conventional methods for applying antibacterial, antifungal or other pesticidal materials to growing vegetation, such as row crops, trees, vines, and ornamental plants, or to cultivated fungi such as mushrooms. They can be applied to plant foliage or to roots or can be otherwise administered to growing vegetation or to seeds or emerging seedlings by delivery to the vegetation or seed or to its vicinity, such as into its growing medium. They can also be applied directly to growing fruits or vegetables to reduce injury to these products caused by plant pathogens. Methods for applying compositions such as these are well known in the art, and are readily adapted to the application of the present compositions.

The compounds or compositions of the invention are applied to vegetation or its locus or to growing fruits or vegetables by conventional methods such as spraying, dusting, or mixing with an irrigation or hydroponic solution that is delivered to the plant. An effective amount of the compositions is readily determined by testing the composition to be administered on a plant or a few plants to determine how much is needed to achieve the desired effect.

For many purposes, a single application may be sufficient. In some situations, multiple applications may be needed to adequately protect the vegetation or plant product of interest. Field survey methods to assess the status of a crop plant or plant product and to determine whether successive treatments are needed are well known to farmers of each particular crop.

A composition of the invention can be admixed with the growing medium for a plant, such as mixing it with a compost or fertilizer or other soil amendment used for growing plants. It can also be admixed with water and applied to plants by aerial or terrestrial spraying methods, by drip, spray or flood irrigation methods, or by hydroponic delivery. These methods are well known in the cultivation of plants and fungi, and adaptation of these methods to the compositions of the invention is within the ordinary skill in the art.

The compositions are sometimes not purified for use: they can be used in crude form after substantially separating the culture fluid from the growing fungi and cellular derived materials, and optionally heating to deactivate remaining fungal material and/or to destroy antifungal and/or nematicidal activity, as when the composition is to be applied to a mushroom, for example. This method of using the crude culture broth, or the culture fluid obtained by removal of the majority of the fungal matter, is also within the scope of the invention. Depending on the method of application, the crude culture fluid in which the Basidiomycete was grown may be used, after substantially removing cellular derived materials and optionally either concentrating it or diluting it with suitable materials such as water or solids.

Optionally, adjuvants such as surfactants, detergents, fertilizers, plant growth regulators, UV blockers, and the like may be added to the compositions of the invention before or when they are applied. In some embodiments, an ammonium salt such as ammonium nitrate or urea ammonium nitrate is added to the mixture before it is applied to the growing vegetation.

The compositions of the invention may be substantially dried prior to use, and may then be applied as a rehydrated solution or suspension, or they may be applied as a solid such as a dust, or they may be mixed with other solids such as clay, sand, vermiculite, compost, or a soil or growing medium of the plants or fungi to be protected. The culture fluid may also be admixed with solids without prior drying, and may then be administered either as a slurry or suspension, or the combination may be dried as by evaporation, and the resultant solids may be applied to growing plants or fungi or proximal to their location to provide the beneficial effects.

Optionally, the compositions of the invention may be applied along with or in a mixture comprising one or more other biocidal or pesticidal materials such as a commercial herbicide, insecticide or fungicide compositions. They may also be applied along with or in a mixture comprising one or more plant growth regulators or growth stimulants. They may also be applied along with or in a mixture comprising one or more fertilizers, especially a fertilizer that provides bioavailable nitrogen, phosphorus, potassium, micronutrients, or iron to a cultivated plant.

The present invention also provides nematicidal compositions that may resemble ones already known (U.S. Pat. No. 6,048,714; U.S. Pat. No. 6,517,851); however, the present invention provides compositions with increased activity due to improved culture conditions that also have the ability to inhibit growth of nematodes on plants, or to inhibit damage to plants or plant products caused by nematodes, by enhancing the resistance of a plant to the growth of or injury by nematodes. These compositions apparently activate the plant's natural defense mechanisms to deter growth of nematodes or modify the defensive responses to reduce the adverse effects of nematodes, even when the population of nematodes is high. Unlike the compositions previously reported, which were designed for soil applications, these compositions are suitably applied to the foliage of emerged plants or to fruit to be protected.

Moreover, because the effect is a plant-mediated one, the desired nematostatic effect does not necessarily require direct contact with the nematodes. Since nematodes typically reside in the soil and affect the roots of a plant, administration of the prior art compositions, which require direct contact with the nematodes to be effective, necessitates delivery directly into the soil. The present compositions may be administered at least in part to the foliage of the plant to provide protection against some of the harmful effects of the nematode infestation. Thus the present compositions may be administered to plants where no symptoms or evidence of nematode infestation are observable, as a nematostatic ‘immune booster’ to protect plants from injury before a damaging infestation develops, and they may be administered by foliar application methods not expected to be effective with a composition that must directly contact the targeted nematodes.

### EXAMPLES

Microbial Strains and Culture Maintenance

Fungal isolates were maintained on potato dextrose agar (PDA, Difco Laboratories, Detroit, Mich.). Bacterial strains, such as Agrobacterium tumefaciens, A. rhizogenes, Acidovorax avenae, Brenneria quercina, Burkholderia cepacia, Erwinia carotovora subsp. carotovora, Erwinia chrysanthemi, Erwinia herbicola, Pantoea herbicola, Pseudomonas corrugata strain 0782-6, Pseudomonas fluorescens, Pseudomonas syringae pv. syringae, Rathayibacter tritici, Xanthomonas campestris pv. campestris strain B-24, and Xanthomonas campestris pv. translucens were also maintained on nutrient agar (NA, Difco Laboratories, Detroit, Mich.).

Ganoderma lucidum and L. sulphureus cultures were maintained in a rich solid medium (RSM) that contained 3 g of yeast extract, 200 g of potato infusion, 20 g of bacto malt extract, 1 g of bacto peptone, 60 g of sucrose, 15 g of bacto agar 1 tsp of molasses, and 3.75 g of ground oat meal per liter water. This culture was used to inoculate the liquid media (broth) cultures. For broth cultures, fungi were grown in PDB, or in rich broth medium (RBM). RBM as used herein contained 15 g of ground oatmeal, 15 g of brewer's yeast, 15 g of corn gluten, 1 tsp of molasses, 2 g of citric acid, and 2 ml of canola oil per liter water. Optionally, another vegetable oil was sometimes added to the medium. All media were sterilized by autoclaving before use.

Optimized Cultural Conditions for Ganoderma and Laetiporus

Surface aeration, pH, and temperature conditions were evaluated to establish the optimum liquid growth conditions for G. lucidum and L. sulphureus. For surface aeration, stationary-flask experiments were performed in 250 ml, 500 ml, 1000 ml, and 2000 ml Erlenmeyer flasks containing 100 ml of RBM. Temperature and pH experiments were then conducted in 2000 ml Erlenmeyer flasks containing 100 ml of RBM. For the pH experiments, pH was adjusted to 3, 4, 5, or 6 by adding either 1N HCl or 1N NaOH. To determine optimum growing temperatures, cultures were grown in 2000 ml Erlenmeyer flasks containing 100 ml of RBM and incubated as stationary cultures at 20°, 25°, 30°, and 35° C. In all experiments, media were sterilized at 121° C. for 20 minutes and inoculated with five one-centimeter plugs (No. 8 cork borer) from a 14 day-old RSM culture grown at 25° C. Flasks for the surface aeration and pH experiments were incubated at 25° C. for 21 days.

### Production of Bioactive Culture Fluids

Once the cultivating conditions for growth of G. lucidum and L. sulphureus were optimized, the following conditions were used to produce bioactive culture fluids. The experiments were performed in 2000 ml Erlenmeyer flasks containing 200 ml of media, thus limiting the depth of the medium to less than about 2-4 inches. Media were autoclaved for 20 minutes at 121° C. After cooling to about 50° C., flasks were inoculated with 5 pieces (cut with a 1 cm diameter No. 8 core borer) of actively growing mycelia from 14-day-old RSM cultures. Flasks were incubated for 21 days at 30° C. After incubation, culture fluids were harvested by centrifugation at 14,000 rpm for 15 minutes at 4° C., and filter sterilized with 0.22 μm filters (Membrane filters, Isopore™, Ireland). Culture fluids were tested for their antimicrobial activity against several bacteria, fungi and nematodes.

Optimum Conditions for Mycelial Growth

Growth conditions of surface area, pH, and temperature of the culture medium affected mycelial growth of G. lucidum and L. sulphureus. Increased surface area of the media/air interface provided by using a larger container for a given volume of medium resulted in increased biomass of both fungi. At 25° C. the greatest amount of mycelial growth occurred with cultures grown in 2000 ml Erlenmeyer flasks after 21 days of incubation for both species of fungi (1005 mg/100 ml for G. lucidum and 1090 mg/100 ml for L. sulphureus). The lowest mycelium concentration was observed in 250 ml flasks (260 mg/100 ml for G. lucidum and 270 mg/100 ml for L. sulphureus). There was no significant difference between 500 ml and 1000 ml Erlenmeyer flasks, which showed similar results and were significantly less than the 2000 ml flask cultures. These demonstrate that a shallow growing medium is advantageous, and typically the medium is not over about 6″ in depth, preferably less than 5″ or less than 4″ in depth. In some embodiments, the medium is at a depth not over 3″.

For larger scale production, typically a large drum such as a 55 gal drum is filled approximately one quarter to half way with medium and is inoculated with the fungal culture to be grown. The medium and growing conditions are as described above. Growth rate of mycelia is increased by suspending plates of e.g. PLEXIGLAS™ into the medium from above.

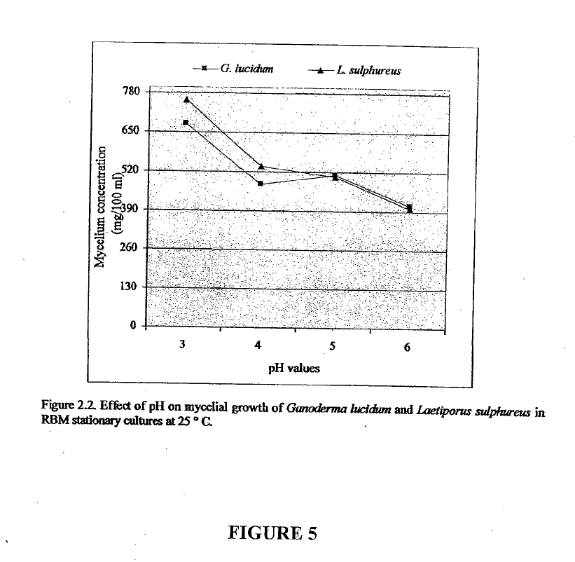

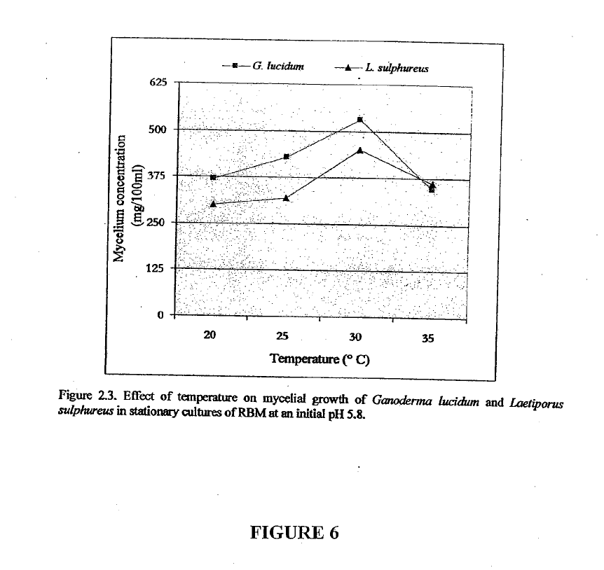

Mycelial growth of both fungi is favored by lower pH's of the culture media (FIG. 5). The greatest amount of mycelial growth was observed at a pH of 3 in both fungi (680 mg/100 ml for G. lucidum and 770 mg/100 ml for L. sulphureus). At media pH's of 4, 5 and 6, mycelial growth was over 30% less than the growth observed at a pH of 3 for both fungi. When grown under different temperatures, optimal growth for both fungi was observed at 30° C. (FIG. 6).

### Bactericidal Activity Determination

Culture fluids were tested for antibacterial metabolites as follows: Centrifuged and filtered sterilized culture media of G. lucidum and L. sulphureus produced in potato dextrose broth (PDB) were mixed 1:1 (v:v) with 2× nutrient agar into 90 mm×15 mm petri plates. Pathogenic bacteria were grown in NA plates at 28° C. for 24 h and bacterial suspensions were performed in a 0.85% saline solution to 10⁷ CFU/ml (O.D. 600=0.5). After agar had cooled down, 15 μl of bacterial suspensions were spotted on the culture filtrate plates. Growth was recorded after 48 hours of incubation at 28° C. as:

+=total growth inhibition;

±=slight growth inhibition;

−=no growth.

### Fungicidal Activity Determination

Centrifuged, filter sterilized culture fluids of G. lucidum and L. sulphureus grown in rich broth medium (RBM) were tested for their fungicidal activity against various plant pathogens. Non-inoculated RBM was used as control in all the experiments. Culture fluids and controls were mixed 1:1 (v:v) with melted 2× PDA and poured into 50×9-mm disposable plastic petri dishes (Falcon®, USA). After agar had cooled down, a single piece (5 mm²) of new actively growing mycelia was placed in the center of the agar plate. Plates were incubated at 25° C. for 5 days. After incubation, the diameter of mycelial colony was measured (in cm) and inhibition of fungal growth determined. Results were reported as the mean percentage of decrease in growth of fungal mycelia after 5 days of incubation.

### Nematicidal Activity Determination

Culture fluids of G. lucidum and L. sulphureus produced in PDB, RBM or in RBM amended with canola or corn oil (RBMCO or RBMVO) were evaluated for their nematicidal activity. In all cases, non-inoculated PDB or RBM (NI-PDB or NI-RBM) were used as controls. Culture fluids produced either in PDB or RBM were used to determine the best incubation time for the production of bioactive nematicidal compounds and at the same time to evaluate the effect of nutrient media on the nematicidal activity. The culture fluids produced in RBM amended with either oil were used to evaluate the effect of oil in the production of nematicidal activity.

Nematicidal activity was assayed by placing 15 J2 juvenile Meloidogyne chitwoodi nematodes into a drop of sterile double distilled water in 3-cm diameter shallow glass dishes. After placing the nematodes in the dishes, treatments were applied in a volume of 800 μl. Plates were incubated at room temperature and results were recorded after 6 h, 12 h, 24 h and 36 h of incubation. The nematicidal activity was reported as the mean percentage of dead nematodes after 36 h. Nematode mortality was evaluated by transferring the nematodes into dishes containing fresh sterile water and incubated for 24 h. Nematodes that did not recover movement were reported to be dead.

Bactericidal Activity Observed

Bactericidal activity was observed in culture fluids of G. lucidum and L. sulphureus from both PDB and RBM. However, osmotic conditions of RBM produced inhibition zones as well so all subsequent antibacterial assays used only PDB cultures. Both G. lucidum and L. sulphureus produced strong inhibition zones against Agrobacterium rhizogenes, A. tumefaciens, Erwinia carotovora subsp. carotovora, and Pseudomonas syringae pv. syringae. No activity was observed against Burkholderia cepacia, Pseudomonas corrugata, Pseudomonas fluorescens, and Xanthomonas campestris pv. translucens. Slight inhibition was observed with Brennaria quercina, and Rathayibacter tritici. Culture fluids that were freshly harvested or stored for 90 days at room temperature had the same amount of activity. Similar results were observed when culture fluids were boiled for an hour, except that no growth inhibition was observed in B. quercina and R. tritici (Table 1) after boiling of the fluid.

TABLE 1
Antibacterial activity of filter-sterilized nutrient broth culture
fluids from Ganoderma lucidum (Gl) and Laetiporus sulphureus
(Ls) are not affected by long term storage (90 days) or boiling
for one hour when compared to freshly harvested culture fluids.

| | 90-day storage | | Boiled | | Non- |
|---|---|---|---|---|---|
| | culture | | culture | | inoc- |
| | fluids | | fluids | | ulated |
| Bacterial species | Gl | Ls | Gl | Ls | NA |
| Acidovorax avenae | +* | ± | + | + | − |
| Agrobacterium rhizogenes | + | + | + | + | − |
| Agrobacterium tumefaciens | + | + | + | + | − |
| Brenneria quercina | ± | ± | − | − | − |
| Burkholderia cepacia | − | − | − | − | − |
| Erwinia carotovora subsp. | + | + | + | + | − |
| carotovora | | | | | |
| Pseudomonas corrugata 0782-6 | − | − | − | − | − |
| Pseudomonas fluorescens | − | − | − | − | − |
| Pseudomonas syringae pv. syringae | + | + | + | + | − |
| Rathayibacter tritici | ± | ± | − | − | − |
| Xanthomonas campestris pv. | + | + | + | + | − |
| campestris | | | | | |
| Xanthomonas campestris pv. | − | − | − | − | − |
| translucens. | | | | | |

Non-inoculated nutrient broth was used as control.
*Bacterial growth inhibition ratings: + = total growth inhibition; ± = slight growth inhibition; − = no growth of test bacterial lawns on PDA plates containing 200 μl of culture fluids applied on a sterile filter paper disk.

Fungicidal Activity Observed

Inhibition of mycelial growth of the test fungi ranged from 0 to 71 percent depending on fungal species tested and appeared to be stable over 90 days of storage at room temperature (Table 2). RBM alone did not inhibit the test fungi. Phoma medicaginis was the most affected of all the fungi tested, while no inhibition was observed with Rhizopus sp. Slight inhibition was observed with Pythium ultimum and Rhizoctonia solani, and the other fungal isolates were moderately inhibited in growth (23% to 54% growth reduction) compared to controls. Boiling for one hour significantly reduced the fungicidal activity in culture fluids of both G. lucidum and L. sulphureus against most fungi. However, growth inhibition of Fusarium sp. Increased modestly with boiled culture fluids from G. lucidum (54%) and remained the same for L. sulphureus (48%) compared to fresh or stored culture fluids. Likewise, an increase in growth inhibition of Rhizoctonia solani was also observed for boiled culture fluids.

TABLE 2
Antifungal activity of filter-sterilized rich broth medium
(RBM) culture fluids from Ganoderma lucidum (Gl) and
Laetiporu sulphureus (Ls) are not affected (similar
to freshly harvested culture fluids) by long term storage
(90 days) but are decreased after boiling for one hour
when compared to freshly harvested culture fluids.
Non-inoculated RMB was used as control.

| | 90-day storage | | | Boiled | | |
|---|---|---|---|---|---|---|
| | of culture fluids | | | culture fluids | | |
| Fungal species | NI-RBM* | Gl+ | Ls | NI-RBM | Gl | Ls |
| Rhizopus sp. | 5.00 | 0 | 0 | 5.00 | 0 | 0 |
| Pythium ultimum | 4.86 | 7 | 10 | 5.00 | 0 | 0 |
| Rhizoctonia solani | 4.70 | 11 | 15 | 4.00 | 38 | 38 |
| Sclerotium sp. | 4.20 | 23 | 23 | 5.00 | 0 | 0 |
| Fusarium sp. | 4.03 | 26 | 49 | 5.00 | 54 | 48 |
| Epicoccum nigrum | 3.43 | 40 | 46 | NT | NT | NT |
| Ascochyta pisi | 3.06 | 46 | 25 | 3.90 | 29 | 29 |
| Alternaria solani | 2.70 | 54 | 57 | 5.00 | 0 | 0 |
| Fusarium sambucinum | 2.47 | 55 | 58 | 3.05 | 30 | 23 |
| Phoma medicaginis | 3.16 | 71 | 61 | 4.75 | 39 | 28 |

+Results are the mean percentage of three experiments of decrease in growth of fungal mycelia (compared to controls) after five days of incubation on assay media composed of cell-free RBM culture fluids: PDA (1:1, v:v).
*Values in NI-RBM are the mean of actual numbers of fungal growth after five days of incubation (columns 2 and 5).

Nematicidal Activity Observed

Both PDB and RBM cell-free culture fluids of Ganoderma lucidum and Laetiporus sulphureus exhibited nematicidal activity against Meloidogyne chitwoodi. Approximately 50% of the nematodes displayed twitchy movements within 1 hour exposure to culture fluids but not when exposed to non-inoculated media. Nematodes that ceased motion and straightened were considered to be dead. Dead nematodes were confirmed by lack of motion 2-4 hour after transferal to fresh water.

Nematicidal activity was detected as early as the 7^{th} day of culture incubation with both G. lucidum and L. sulphureus (Table 3). With G. lucidum and L. sulphureus, the highest level of nematicidal activity was observed with culture fluids from 28, 21, and 14-day-old PDB cultures with a slight decrease in activity with 35-day-old cultures. A similar trend was observed for both fungi in RBM. However, levels of activity were much higher in RBM than in PDB.

TABLE 3
Nematicidal activity of culture fluids from
Ganoderma lucidum and Laetiporus sulphureus grown in
potato dextrose broth (PDB) and rich broth medium (RBM).
Culture fluids were collected at 7-day intervals for up to 35
days and screened against J2 juvenile Meloidogyne chitwoodi.

| | Culture fluids | | | | | | | Non-inoculated | | |
|---|---|---|---|---|---|---|---|---|---|---|
| Incubation | G. lucidum | | | | L. sulphureus | | | controls | | |
| time (days) | PDB | | RBM | | PDB | RBM | | NI-PDB | NI-RBM | |
| 7 | 40 | c + | 52 | c | 36 e | 61 | c | 0.6 e | 0 | b |
| 14 | 77 | a | 90 | b | 74 b | 91 | b | 1.6 b | 0 | b |
| 21 | 77 | a | 100 | a | 78 a | 100 | a | 1.3 c | 0.33 | a |
| 28 | 79 | a | 100 | a | 71 c | 100 | a | 2.0 a | 0 | b |
| 35 | 68 | b | 99 | a | 66 d | 99 | a | 1.0 d | 0 | b |

+ Means within each column followed by the same letter are not significantly different at p < 0.05 (LSD). Numbers are mean percentage of dead nematodes after 36 h.

Nematicidal Activity Produced in RBM Amended with Canola Oil or Corn Oil

In both PDB and RBM media, oil droplets were observed in cultures of G. lucidum and L. sulphureus. In PDB cultures, oil droplets remained to the conclusion of each experiment, but visible oil droplets had disappeared in RBM by day 30. To test the hypothesis that oil may serve as a nutrient source for these fungi, RBM was amended either with 1% of canola oil or 1% of corn oil (RBMCO and RBMVO, respectively). Nematicidal activity was observed in all the culture fluids produced by G. lucidum and L. sulphureus in RBM, RBMCO, and RBMVO but not in RBM controls. However, nematicidal activity of culture fluids from both oil supplemented media with both fungi were lower than non-supplemented media.

### Example 2

### Preparation of Compositions for Application to Plants

The compositions of the invention can be prepared for administration to plants by filtration to remove solids followed by dilution with water. Typically, additional purification is used to remove substantially all cellular derived material. This may be achieved by centrifugation, sedimentation, gel filtration, or dialysis, for example, to remove cellular debris. Using these or other conventional methods, materials having a molecular weight above about 100,000 may be employed. Additional processing and purification steps such as, for example, treatment with activated carbon or charcoal to remove lipophilic materials and/or colored materials may be employed. Similarly, the aqueous solution may be extracted with a water-immiscible organic solvent to remove lipophilic materials.

Optionally, surface active agents may be added to increase effectiveness for application to foliage or fruits. Other nutrients and adjuvants such as urea ammonium nitrate (UAN) known to enhance the effectiveness of plant protective agents may also be added.

Optionally, each batch of product (partially purified solution, ‘compost tea’, or concentrate, for example) can be tested for a desired bioactivity or profile of activities, or for the presence of specific compound or compounds associated with desired activities. Each batch can then be concentrated or diluted as needed to provide a standardized level of bioactivity before distribution to a user.

### Example 3

### Application to Plants

The partially purified composition described above is then directly applied to plants or parts of plants to be protected by conventional application methods and equipment, to reduce infestations of plant pathogens that could damage the plants or plant products. For example, the composition may be diluted to a convenient volume for spraying, and can be sprayed onto the plants, soil or both. Conventional methods for determining the amount to be used are well known, and can be used to guide the user in determining what degree of dilution is appropriate to control the targeted pests while avoiding harm to the vegetation or crop.

Alternatively, the compositions may be used as a soil drench or spray, or they may be prepared as a solid formulation by concentration, and then they may be incorporated into the soil around newly planted seeds, emerging seedlings, or established plants. They may also be applied during planting as a band treatment in the vicinity of the planted seeds or transplanted seedlings, which permits the composition to be incorporated into the soil that immediately surrounds the sensitive seedlings.

Alternatively, they may be used as a seed treatment prior to planting the seeds, in which case the seeds may be coated with a dried form of the composition or they may be soaked in a solution comprising an active component obtainable from the compositions of the invention, such as a concentrated culture fluid.

### Example 4

### Application to Preserve Wood

The compositions of the invention may be used to preserve wood by reducing damage caused by termites, fungi or both. A composition of the invention can be prepared at a concentration up to about 30 brix, and may be applied directly to wood to be protected. Optionally, a pressure treatment may be used to increase uptake of the composition into the treated wood, as is well known in the art for other types of wood preservatives.

### Example 5

### Use to Control Non-Plant Pathogens

A Plasmodium culture (malaria protozoans) in a petri dish was treated with a 2000:1 dilution of a composition of the invention prepared from L. sulphureus, and concentrated to a 30 brix concentrate, using a ‘short boil’ process. At this dilution, about 50% of malarial protozoans were killed within a few hours. Higher doses provided increased toxicity to the protozoan population.

### Example 6

### Improved Fungal Culture Growing Methods

Cultures of L. sulphureus were grown in PYREX® containers both with and without an added piece of polyurethane foam to provide additional growing surface. It was estimated that the foam tripled the growing surface area relative to the PYREX® container alone. The concentration of sugars in the growing medium was tracked for two months by periodically measuring the brix of the medium. Brix is a parameter used to monitor sugar content in growing grapes, and indicates the approximate concentration of dissolved solids in an aqueous mixture. The culture with the added polyurethane foam consumed sugars from its medium at about three times the rate of the culture without the added polyurethane foam, indicating that it was growing roughly three times faster due to the added growing surfaces provided by the polyurethane foam.

The detailed description and examples provided herein serve to illustrate the invention, not to limit it. Variations and combinations of the features of the invention will be obvious to the ordinary practitioner from the description provided, and these variations and combinations are also within the scope of the invention.

1-49. (canceled)

50. A method of producing a composition having biocidal activity against a pathogen, which method comprises culturing a Basidiomycete fungus on a substantially liquid growth medium to obtain a culture,
harvesting culture fluid from said culture;
removing substantially all cellular debris from said culture and/or
removing colored materials, and/or removing lipophilic materials, and/or
removing materials having a molecular weight above about 100,000.

51. The method of claim 50, wherein the Basidiomycete fungus is cultured at a pH below 4,
wherein the liquid medium comprises rich broth medium or potato dextrose broth,
and wherein additional growing surface area is provided in addition to the growing surfaces provided by the container used to hold the culture.

52. The method of claim 50 wherein the Basidiomycete fungus is a Ganoderma or Laetiporus species.

53. The method of claim 50, wherein the Basidiomycete fungus is grown at a temperature of about 30° C. for two to five weeks.

54. A bactericidal composition comprising a heat-stable oligosaccharide comprising D-glucose, D-ribose and D-galactofuranose having a molecular weight between about 2000 and about 5000, which oligosaccharide is produced by a fungus of a Basidiomycete species and is active against at least one bacterium, and
which composition is free of cellular debris derived from the fungus.

55. The bactericidal composition of claim 54 which is further purified by removal of colored materials, and/or by removal of lipophilic materials, and/or by removal of materials having a molecular weight above about 100,000.

56. The bactericidal composition of claim 54, wherein the Basidiomycete species is a Ganoderma or Laetiporus species.

57. The bactericidal composition of claim 54, wherein the composition is active against a phytopathogenic bacterium comprising at least one species selected from the group consisting of Agrobacterium, Acidovorax, Brenneria, Erwinia, Pantoea, Pseudomonas, and Xanthomonas.

58. A nematicidal or antifungal composition derived from a Basidiomycete fungus comprising a carbohydrate having a molecular weight between about 2000 and 5000, that is stable to treatment with a protease and is denatured upon boiling in aqueous solution,
wherein the composition is substantially free of cellular debris of said fungus.

59. The composition of claim 58, which is substantially free of colored materials, and/or lipophilic materials, and/or materials having a molecular weight above about 100,000.

60. The composition of claim 58 that is produced by a Ganoderma or Laetiporus species.

61. A method of to reduce the adverse effects of a bacterium on a substance, composition, plant, or plant product, which method comprises applying an effective amount of a composition according to claim 54 to the substance, composition, plant or plant product to be protected or to the locus thereof.

62. The method of claim 61 wherein the plant product is wood.

63. A method to treat or prevent an infection of an animal by a bacterium which comprises administering to an animal in need of such treatment an effective amount of the composition of claim 54.

64. A method of to reduce the adverse effects of a nematode or fungus on a substance, composition, plant, or plant product, which method comprises applying an effective amount of a composition according to claim 61 to the substance, composition, plant or plant product to be protected or to the locus thereof.

65. The method of claim 61 wherein the plant product is wood.

66. A method to treat or prevent an infection of an animal by a nematode or fungus which comprises administering to an animal in need of such treatment an effective amount of the composition of claim 61.

67. An improved method of producing a culture fluid by growing a fungus on an aqueous medium in a large container, wherein the improvement comprises:
providing additional growing surfaces suitable to support mycelial growth in addition to the surfaces of the container,
wherein the total surface area/volume ratio of the medium-wetted surfaces of the container plus the additional growing surfaces is at least about 50 cm²/liter of medium,
and/or wherein the additional growing surfaces provide growing surface area that is at least about 50% of the growing surface area provided by the medium-wetted surfaces of the container.
